(19) United States
(12) Patent Application Publication
(10) Pub. No.: US 2009/0234279 A1
(43) Pub. Date: Sep. 17, 2009

(54) MECHANICAL PROPULSION CATHETER APPARATUS AND METHODS

(76) Inventor: James A. Goldstein, Bloomfield Hill, MI (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 12/404,854

(22) Filed: Mar. 16, 2009

Related U.S. Application Data
(60) Provisional application No. 61/036,714, filed on Mar. 14, 2008.

(51) Int. Cl. A61M 25/092

(52) U.S. Cl. 604/95.01

(57) ABSTRACT

Medical devices such as catheter-based technologies for therapeutic interventions in the coronary arterial circulation and other vascular beds are presented. Apparatus, systems and methods for improving the performance and/or operability of catheter navigation through a vascular pathway are presented. Deliverability of vascular therapeutic devices is improved through mechanical catheter control systems.

### CROSS-REFERENCE TO RELATED APPLICATIONS

This application claims priority from U.S. Provisional Application Ser. No. 61/036,714, filed on Mar. 14, 2008, which is incorporated herein by reference in its entirety.

### FIELD OF THE INVENTION

The present invention relates generally to medical devices such as catheter-based technologies for therapeutic interventions in the coronary arterial circulation and other vascular beds. More specifically, the present invention relates to apparatus, systems and methods for improving the performance and/or operability of catheter navigation through a vascular pathway.

### BACKGROUND

Catheter-based technologies for therapeutic interventions in the coronary arterial circulation and other vascular beds (e.g. cerebrovascular, etc) are widely employed for intravascular therapy for atherosclerotic and other types of vascular disease. One commonly employed technological approach to vascular atherosclerotic disease is the utilization of balloon dilatation catheters advanced over fine guide wires introduced into specific vessels in the coronary artery circulation. The balloon catheters are advanced over wires and inflated at points of obstruction. The balloon catheters are also utilized to deliver and deploy stents, intra-arterial prostheses which scaffold such obstructions, resulting in improved longer term procedural success.

To advance the balloon and deliver the stent, the catheter and device must traverse an arterial network typically containing atheromatous plaques and vessel tortuosity, which together constitute resistance to advancement of such catheters. These techniques are predominantly performed percutaneously, that is catheters are inserted and maneuvered from outside of the body with manipulation over a long length of catheter shaft with the intent to deliver the distal end of the balloon dilatation catheter (often with an associated mounted stent) far down-stream to the target lesion in the circulation. Owing to the distance traveled from outside the body to the distal target circulation, combined with the tortuous nature of blood vessels and the resistance imposed by obstructions within diseased circulations, the ability to deliver the catheter device (balloon often with superimposed mounted stent) to the target may be difficult, as the ability to transmit the pushing force from the operator's hand outside of the body to the downstream (distal) device may be limited.

A guide catheter is inserted from outside a patient's body, into a peripheral vessel and serves as the accessibility conduit to inject contrast dye and deliver devices to the desired vascular location. A certain amount of force must be applied to the stent catheter systems to cross obstacles. The guide catheter also provides and maintains rigidity of the system which helps minimize kinking of the catheter when it comes into contact with an obstacle.

There are essentially two types of catheter systems: “Over-the-wire” (OTW) and Rapid Exchange (Monorail) catheters. Both catheter types cooperate with guide wires for use in placement of the catheter in the vasculature of the patient. The first type of catheter is an “over-the-wire” catheter in which a guide wire lumen extends the full length of the catheter shaft. This type of catheter requires that the guide wire be sufficiently long for its proximal end to remain in place until the distal end of the catheter has been withdrawn from the patient. The second type of catheter is so-called “rapid-exchange” catheter in which the guide wire lumen does not extend the full length of the catheter shaft. Instead, the guide wire exits the catheter shaft at the distal end of the catheter and at some point between the distal end and the proximal end of the catheter which is preferably closer to the distal end. Thus, when positioned in the vasculature, most of the guide wire extends externally in a proximal direction alongside of the catheter shaft. This limits the interaction between the guide wire and reduces the necessary extracorporeal length of the guide wire in comparison to the guide wire length required by over-the-wire systems. Rapid-exchange catheters typically have multiple separate lumens with one lumen dedicated as a guide wire lumen which can be used for the exchange of catheters. Both types of catheter systems are utilized to deploy stents. Detailed descriptions of these systems follows.

### Equipment

An OTW catheter is used in conjunction with a separate guide wire to cross a narrowed site in a person's vascular system. The structure of the OTW catheters is such that the guide wire lumen extends the full length of the catheter shaft. In general, the shaft of such catheters typically contain two separate lumens, one of which accommodates the guide wire while another transmits fluid and hydraulic pressure along the length of the catheter. The proximal end of the catheter has an adapter to interface with the source of hydraulic pressure while the distal end includes a balloon for dilatation of the artery or other vessel. This type of catheter requires that the guide wire be sufficiently long for its proximal end to remain in place until the distal end of the catheter has been withdrawn from the patient. OTW angioplasty balloon catheters typically have a coaxial construction with an inner tube which defines an inner lumen and an outward tube coaxially disposed about the inner lumen to define an outer lumen between the walls of the inner and outer tubes. The inner lumen typically will have a guide wire running there through, while the outer lumen conveys inflation fluid from the proximal end of the catheter to the inflatable balloon.

A second type of catheter is the “rapid-exchange” catheter, a design in which the guide wire lumen does not extend the full length of the catheter shaft. Instead, the guide wire exits the catheter shaft at the distal end of the catheter and at some point between the distal end and the proximal end of the catheter which is preferably closer to the distal end. Thus, when positioned in the vasculature, most of the guide wire extends externally in a proximal direction along side of the catheter shaft. This limits the interaction between the guide wire and reduces the necessary extracorporeal length of the guide wire in comparison to the guide wire length required by over-the-wire systems. Rapid-exchange catheters typically have multiple separate lumens with one lumen dedicated as a guide wire lumen which can be used for the exchange of catheters.

One advantage of the OTW coaxial design over the Monorail catheter design is that the proximal portion of the outer tube can be formed from a relatively stiff material to provide increased “pushability” to the catheter. Another advantage is that the outer and inner tubes can be narrowed in the distal region of the catheter and under the proximal waist of the balloon to reduce the distal shaft diameter and the profile of the catheter in its deflated state. A further advantage is that the coaxial design is symmetrically flexible in all directions. In addition, the coaxial catheter allows some degree of relative movement to take place between the inner tube and the outer tube when the catheter is bent which also increases the flexibility.

Stents are tubular metal prostheses employed for treatment of disease in the coronary and cerebrovascular circulations, circulation of the kidneys and other vascular beds throughout the body to expand the arterial lumen and maintain the patency of the vessel. Stenting has become an increasingly important treatment option for patients with coronary artery disease and the number of percutaneous coronary interventions (PCI) with stent implantation is rapidly increasing. Stents are delivered to the coronary artery using long flexible vascular catheters typically inserted through a femoral artery. For balloon expandable stents, a balloon on the delivery catheter is expanded which expands and deforms the stent to the desired diameter where upon the balloon is deflated and removed. For self-expanding stents, the device is prepared in a constrained position and shape, deployment achieved by release from the delivery catheter by pulling back a restraining sheath, allowing the resiliently expanding stent to come into engagement with the vessel wall.

### General Performance Characteristics of Catheters

“Crossing profile” of a catheter refers to the cross-sectional profile of the balloon when deflated, while “shaft profile” refers to the dimensions of the cross-sectional profile of the catheter shaft communicating with the balloon. Smaller or “lower profile” catheters provide several advantages.

“Conformability” is a property that describes the ability of the coronary stent systems to conform to the geometrical shape of an artery.

Stent catheter systems can be measured with respect to their “stiffness.” A certain amount of force must be applied to the stent catheter systems to cross obstacles. Stiffness provides a measure of the force that needs to be applied to a stent-catheter system in order to navigate the curvature required to negotiate a particular curve in a vessel.

“Pushability” (or column strength) of an angioplasty system varies as a function of the compliance of the supporting element. For the same material of construction, catheters having thinner shaft walls are less pushable, and thus more prone to axial compression, particularly during introduction across critical lesions. Over-the-wire systems rely upon the catheter shaft for column support whereas non-over-the-wire systems rely upon the guide wire for their support. The pushability varies as a function of the rigidity and thickness of the material used in the walls of the catheter shaft. Accordingly, reducing the shaft wall thickness to reduce the shaft profile, without substituting a more rigid material, adversely effects the pushability of these systems and thus the clinical utility of these devices in the treatment of high grade lesions. Although this disadvantage has been partially offset by the use of increasingly rigid plastics for the shaft walls, increasing rigidity is only beneficial to a limited extent.

“Steerability” refers to performance of guide wires, whose directional control varies directly as a function of the profile of the guide wire mandrel and inversely as a function of a friction that develops in response to rotation of the guide wire relative to the catheter. The amount of friction that develops in this circumstance varies as a function of: (1) The normal force between the catheter and guide wire during inter-component rotation and (2) the coefficient to friction of the catheter guide wire interface. The magnitude of the catheter-guide wire contact surface area varies inversely as a function of the catheter-guide wire clearance. Reducing the profile of the catheter shaft reduces the clearance for the guide wire which inevitably compromises the steerability of the composite system.

“Trackability”. Both flexibility and trackability are essential to stent delivery. To implant a stent, the device must have first gone through an arterial network presenting atheromatous plaques and tortuosities which impose resistance to advancement of the catheter. Trackability measures the force that needs to be transmitted to a device system to cross a particular vessel segment. Trackability describes the capacity of a stent catheter system to advance distally over a guide wire along the course of a vessel, even in the case of narrow and tortuous vessels, particularly with respect to bends to negotiate curves or angles (flexibility). Trackability, or the ease with which the catheter component can be advanced over the guide wire, varies as a function of the flexibility of the distal aspect of the catheter and the magnitude of linear resistance that develops between these system components during coaxial movement of one component relative to the other. The linear resistance that develops in this circumstance varies as a function of the magnitude of the contact surface area between the catheter and guide wire and the coefficient of friction of the catheter-guide wire interface. Reducing the catheter-guide wire clearance, with the aim to reduce the shaft profile, increases the catheter guide-wire contact surface area and thus compromises the tractability of the system.

Over the years, there has been substantial improvements in catheter technology designed to facilitate the delivery of catheters and their associated therapeutic devices (e.g. stents) to various aspects of the circulations targeted for therapy. These catheter shafts typically are constructed with fine spiraled or braided metallic or non-metallic strands of reinforcement material embedded in thin cylindrical walls of flexible tubing. For proper operation, these types of catheters require certain performance characteristics. These catheters should transmit force from proximal and outside of a patient to distal and inside of the patient for precise positioning. Such catheters should prevent collapse, kinking or alteration of inner lumen and be able to contain fluid pressures of up to 1,000 PSI. These catheters should also be relatively stiff at the proximal end for optimal “pushability” and progressively more flexible towards the distal end to be able to maneuver through tortuous vessels without damaging the vessel wall. Reinforcement of the tubing structure is necessary when the tubing is required to withstand a variety of different mechanical stresses such as torque, pushing, pulling, pressure and sharing forces. Furthermore, such catheters should provide the largest inside diameter and give an outside diameter i.e., to have the thinnest wall possible without compromising catheter performance. It is desirable that such catheters have a lubricous inside surface for easy insertion of therapeutic devices.

Much of the progress that has been achieved in the development of lower profile angioplasty dilatation balloon catheter systems, and particularly “over-the-wire” angioplasty catheter systems, has resulted from miniaturization. Considerable effort has been directed toward the development of these systems with progressively lower cross-sectional shaft and “crossing” profiles. They provoke less trauma during introduction within the vascular system. Catheters with lower “crossing profiles” require less effort to manipulate across severe obstructions than devices of larger crossing profile. Recent developments in plastics allow manufacture of the shaft and balloon components of these devices with thinner walls than previously possible. Other new technologies allow the construction of these devices with smaller caliber channels. Unfortunately the practice of miniaturization provides diminishing returns. Miniaturization adversely affects the pushability, hydraulic performance, steerability and trackability of angioplasty catheter systems.

Currently used catheter stent delivery systems do not fully meet physicians expectations. Many stent delivery catheters suffer from inflexibility and high cross-section profiles, which hamper endovascular positioning. Various technological improvements have been made to the guiding catheters, wires and balloon dilatation catheter-stent delivery systems to improve the pushability, flexibility and trackability of such devices in order to improve the overall deliverability to target circulations. Despite these advances, there are still a substantial number of cases in which the nature of the patients' target circulation limits the deliverability of the therapeutic device to the intended target, thereby resulting in more prolonged procedures, utilization of additional expensive catheter equipment, and sometimes, procedural failure. In many cases, delivery of stents to target lesions remains difficult, occasionally impossible. Current stent delivery systems suffer from a number of drawbacks, including poor tracking, especially with longer stents, particularly when they must be advanced through tortuous, diseased vessels that are stiff, narrowed and impose significant difficulty in the advancement of catheters and delivery of balloon-stent systems. Also, many stent delivery catheter systems suffer from inflexibility and high cross-sectional profiles, which hamper endovascular positioning.

There is a long-felt, unmet need for stent delivery systems and stents with improved delivery performance characteristics with respect to pushability, trackability and flexibility, which together would enhance endovascular positioning through tortuous vascular pathways. An improved catheter system with enhanced pushability, trackability may also allow reduction in catheter profile. These concepts may be particularly applicable to recently introduced robotically-controlled catheter systems (e.g. Stereotaxis, Inc., Hansen, Inc., and Corindus, Inc.) which deliver and manipulate catheters by computer-machine interface without a human hand directly interacting with the catheter system. These non-human catheter manipulations may also be limited owing to difficulty maneuvering the catheters through challenging vascular terrain without the direct aid and control of the human hand. Furthermore, as catheter-based therapies are applied to more demanding anatomic circulations, such as the cerebral circulation in which vessels may be very small, tortuous, delicate and the targets sites for device delivery very far downstream, catheter systems that can smoothly navigate these terrains may be very attractive, especially if miniaturized.

### SUMMARY OF THE INVENTION

Objects of the instant invention include to provide systems and methods to improve the deliverability of vascular therapeutic devices delivered manually and/or by robotic or other non-manual mechanical catheter control systems. The foregoing and other objects are intended to be illustrative of the invention and are not meant in a limiting sense. Many possible embodiments of the invention may be made and will be readily evident upon a study of the following specification and accompanying drawings comprising a part thereof. Various features and subcombinations of invention may be employed without reference to other features and subcombinations. Other objects and advantages of this invention will become apparent from the following description taken in connection with the accompanying drawings, wherein is set forth by way of illustration and example, an embodiment of this invention and various features thereof.

One or more preferred embodiments of the invention, illustrative of the best mode in which the applicant has contemplated applying the principles, is set forth in the following description and is shown in the drawings and is particularly and distinctly pointed out and set forth in the appended claims. A more complete appreciation of the invention and many of the advantages thereof will be readily obtained as the same becomes better understood by references to the detailed description when considered in connection with the accompanying drawings.

### BRIEF DESCRIPTION OF THE DRAWINGS

One or more embodiments are set forth in the following description and is shown in the drawings and is particularly and distinctly pointed out and set forth in the appended claims.

FIGS. 1(a-e) show cross-sectional views of a mechanical propulsion catheter apparatus with a single accordion spring segment at the end proximal to a user. FIG. 1a shows the spring segment in a disengaged, fully extended position. FIG. 1b shows the catheter tip after encountering an obstacle and, as additional force is exerted linearly along the length of the catheter from the proximal end to the distal end, the spring segment is compressed. FIG. 1c shows the catheter tip being thrust forward and through the blockage by the decompressing spring segment. FIG. 1d shows the catheter tip being stopped, again, by the obstacle while the spring segment is compressed, again. FIG. 1e shows the catheter after it has been thrust forward and through the blockage by the decompressing spring segment.

FIG. 2a shows a portion of an elongated shaft of a mechanical propulsion catheter that extends, generally, from a proximal end of a spring segment to an end proximal to the user. FIG. 2b shows a cross section of the elongated shaft segment.

FIG. 3a shows a portion a spring segment of a mechanical propulsion catheter with a single accordion spring segment that extends from an inflation lumen at the distal end of the catheter to the distal end of an elongated shaft segment. FIG. 3b shows a cross section of the spring segment.

FIG. 4a shows a portion of an inflation lumen located at the distal end of a mechanical propulsion catheter. FIG. 4b shows a cross section of the inflation lumen segment.

FIGS. 5(a-d) show cross-sectional views of a mechanical propulsion catheter apparatus with two accordion spring segments. FIG. 5a shows a proximal spring segment in a disengaged, fully extended position and a distal spring segment that has been compressed as the catheter tip presses against an obstacle. FIG. 5b shows the catheter tip being thrust forward and through the blockage by the decompressing distal spring segment as the proximal spring segment compresses. FIG. 5c shows the catheter tip continuing to be thrust forward and through the blockage by the decompressing proximal spring segment and the compressing distal spring segment. FIG. 5d shows the catheter tip and distal spring segment fully passed through the blockage and continuing to be thrust forward and through the blockage by the decompressing proximal spring segment.

FIGS. 6(a-d) show cross-sectional views of a mechanical propulsion catheter apparatus with a single accordion spring segment located at the distal tip. FIG. 6a shows a spring segment that has been compressed as the catheter tip presses against an obstacle. FIG. 6b shows the catheter tip being thrust forward and through the blockage by the decompressing spring segment. FIG. 6c shows the catheter tip continuing to be thrust forward and through the blockage by compressing spring segment. FIG. 6d shows the catheter tip and spring segment fully passed through the blockage.

FIGS. 7(a-d) show cross-sectional views of a mechanical propulsion catheter apparatus with two accordion spring segments. FIG. 7a shows a proximal spring segment in a disengaged, fully extended position and a distal spring segment that has been compressed as the catheter tip presses against an obstacle. FIG. 7b shows the catheter tip being thrust forward and through the blockage by the decompressing distal spring segment as the proximal spring segment compresses. FIG. 7c shows the catheter tip continuing to be thrust forward and through the blockage by the decompressing proximal spring segment and the compressing distal spring segment. FIG. 7d shows the catheter tip and distal spring segment fully passed through the blockage and continuing to be thrust forward and through the blockage by the decompressing proximal spring segment.

FIG. 8a shows a portion of an elongated shaft of a mechanical propulsion catheter that extends, generally, from a proximal end of a spring segment to an end proximal to the user. FIG. 8b shows a cross section of the elongated shaft segment.

FIG. 9a shows a portion a spring segment of a mechanical propulsion catheter. FIG. 9b shows a cross section of the spring segment.

FIG. 10a shows a portion of a mechanical propulsion catheter just proximal of the distal tip. FIG. 10b shows a cross section of this segment.

FIG. 11 shows a portion of a mechanical propulsion catheter with two spring segments in series.

FIGS. 12(a-d) show cross-sectional views of a mechanical propulsion catheter apparatus with a dual accordion spring segments. FIG. 12a shows both spring segments in disengaged, fully extended, positions as the catheter tip presses against an obstacle. FIG. 12b shows both spring segments in a compressed position. FIG. 12c shows one spring segment in a compressed position and the other spring segment in a disengaged, fully extended position. FIG. 12d shows the catheter tip fully passed through the blockage with both spring segments returned to the disengaged, fully extended position.

FIG. 13 shows a compressible soft wheel cylinder with rolling, floating, soft, deflecting spherical bearings.

FIG. 14 shows a compressible soft wheel cylinder with rolling, floating, soft, deflecting spherical bearings.

FIG. 15 shows a compressible soft wheel cylinder with rolling, floating, soft, deflecting spherical bearings.

FIG. 16 shows a compressible soft wheel cylinder with rolling, floating, soft, deflecting spherical bearings located at proximal and distal ends of a balloon-stent delivery segment.

FIG. 17 shows a compressible soft wheel cylinder with rolling, floating, soft, deflecting spherical bearings expanding outwardly and sliding over the top of a balloon-stent delivery segment.

FIG. 18 shows a compressible soft wheel cylinder with rolling, floating, soft, deflecting spherical bearings expanding outwardly and sliding over the top of an elongated shaft segment.

FIG. 19 shows a cross section of a compressible soft wheel cylinder with rolling, floating, soft, deflecting spherical bearings.

### DETAILED DESCRIPTION

As required, one or more detailed embodiments of the present invention are disclosed herein; however, it is to be understood that the disclosed embodiments are merely exemplary of the principles of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Preferred embodiments of the present invention include a balloon dilatation catheter (and catheter balloon-stent delivery system) with force-multiplying springs designed to, when loaded (engaged) or unloaded (disengaged), release energy from the spring action which generates forward propulsive force to facilitate advancement of the catheter along a vascular route over a guide wire.

In preferred embodiments, the catheter is constructed in standard lengths of 100 to 120 centimeters and may be executed in both over-the-wire (OTW) and monorail type systems. In some preferred embodiments, the balloon catheter of the system generally compromises an elongated shaft having a proximal end, distal end, an inflation lumen, extending from the proximal end to a location proximal of the distal end of the shaft and a guide wire lumen extending from a location distal of the proximal end to the distal end of the shaft. A proximal shaft section comprises a proximal tubular member having a proximal end, distal end and defining a proximal section of the inflation lumen. At its distal end, the catheter has a standard inflatable balloon in continuity with an inflation hydraulic lumen suitable for dilatation of coronary obstructions as well as to serve as a stent delivery system, as with traditional balloon dilatation catheter systems. A distal shaft section comprises an outer tubular member having a proximal end secured to the distal end of the proximal shaft section and having a distal end. The distal shaft section comprises a distal section of the inflation lumen in fluid communication with the proximal section of the inflation lumen. The guide wire lumen defined by the inner tubular members include communication with the guide wire distal port at the distal end of the catheter and a guide wire proximal port distal to the proximal shaft section.

The system further comprises at least one spring. The at least one spring may be disposed proximally or distally, or in dual proximal-distal configurations. The springs are made of metallic or other suitable materials with variable spring tension, which may be varied to form a family of catheters with different spring propulsive characteristics based on the type of metal and the mechanical design of the spring.

Other preferred embodiments comprise a vascular catheter comprised of an inner guide wire lumen, an outer balloon inflation lumen with the catheter constructed either in a traditional rapid exchange or an over-the-wire configuration. The system further comprises an accordion spring section that may be disposed either: (1) Proximal to the balloon segment, or; (2) both proximal and distal to the balloon segment.

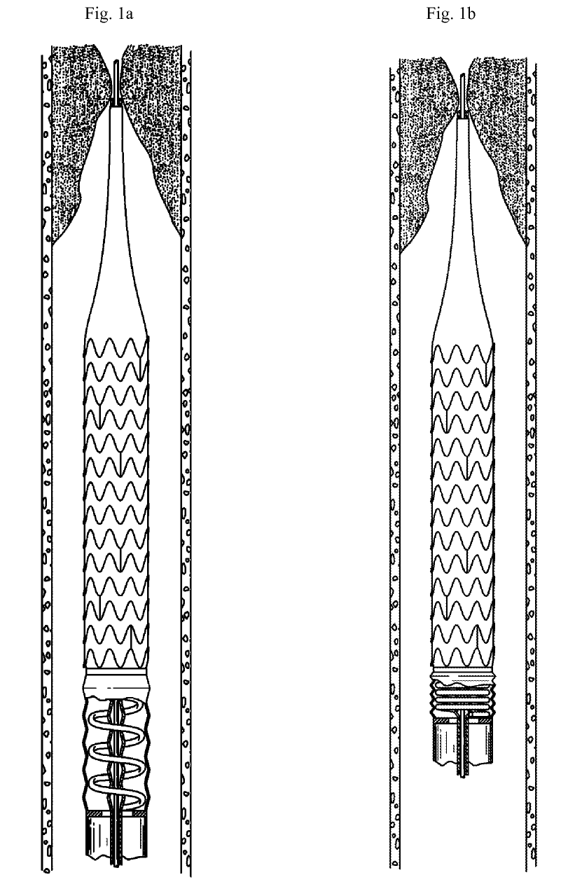

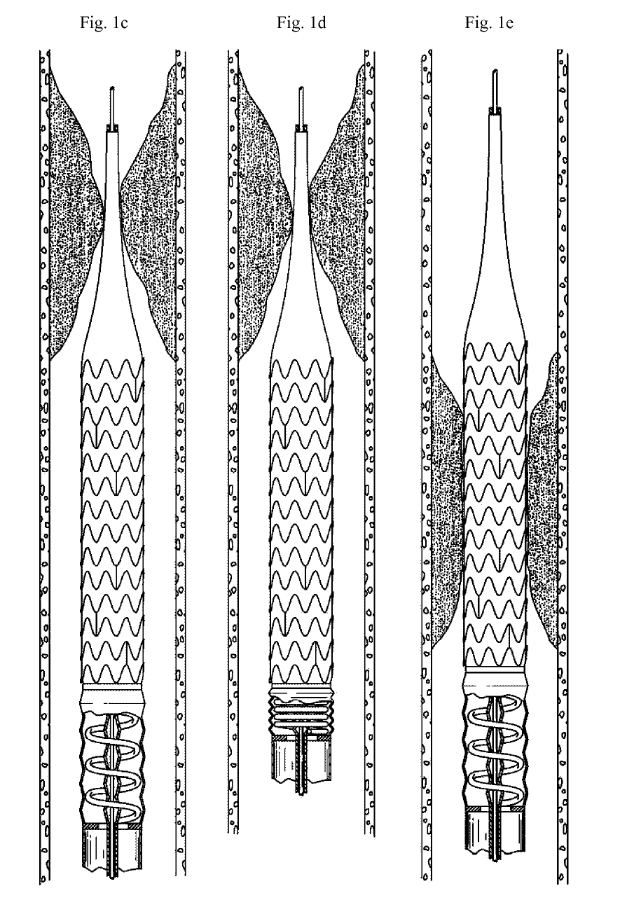

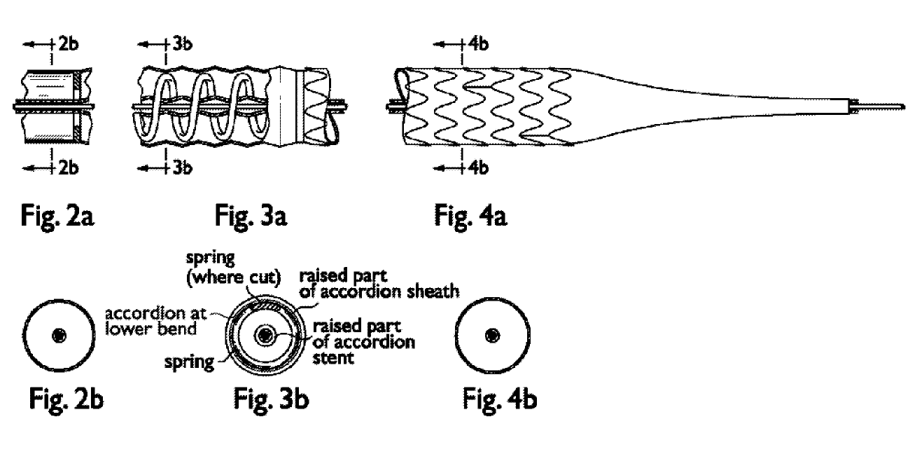

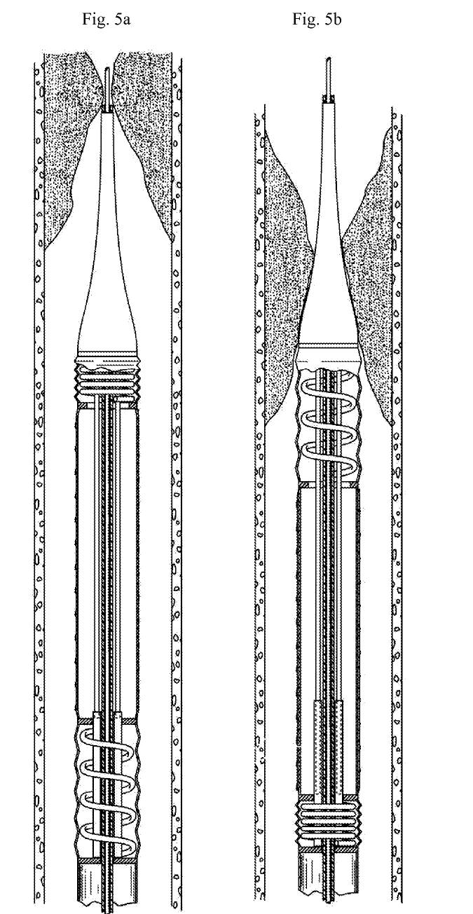

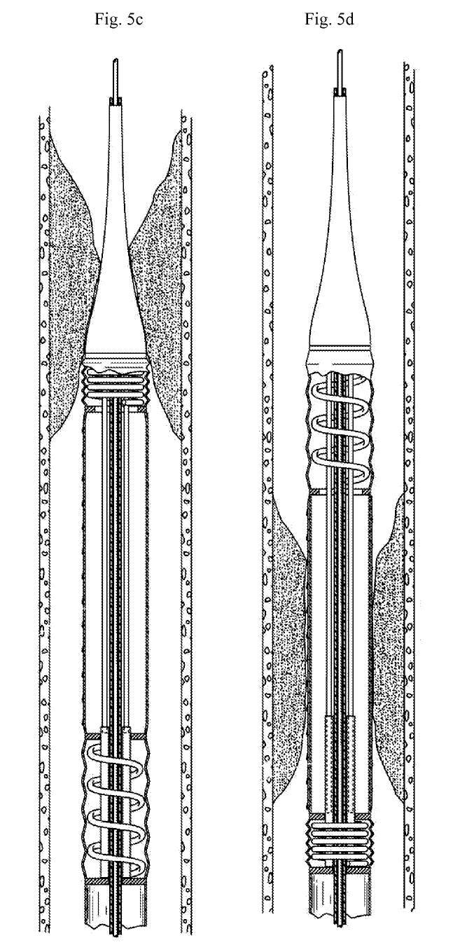

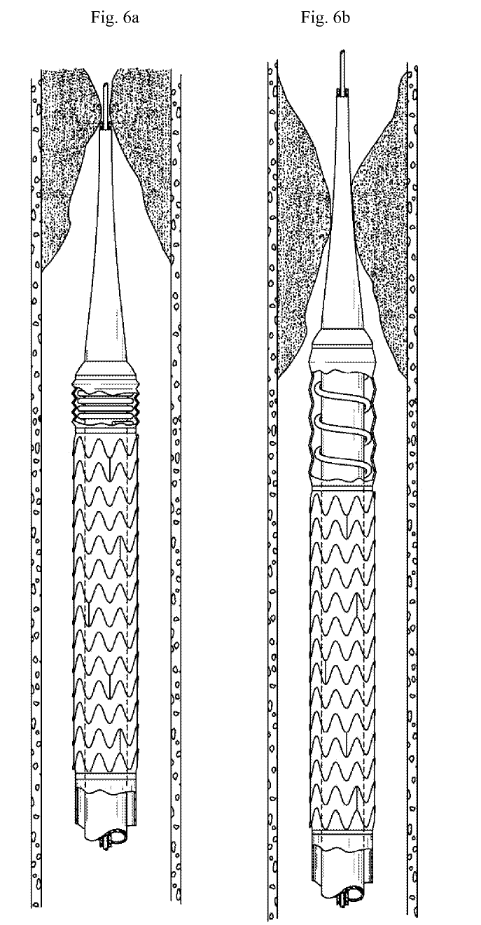

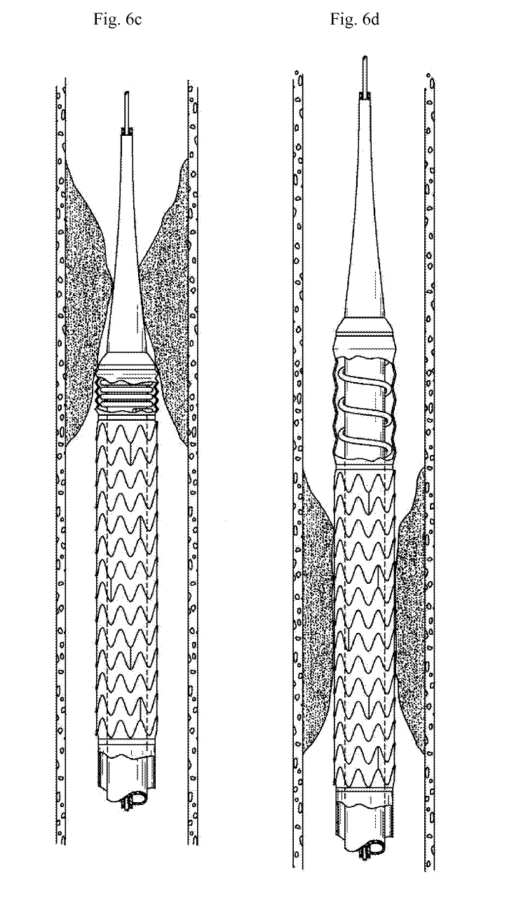

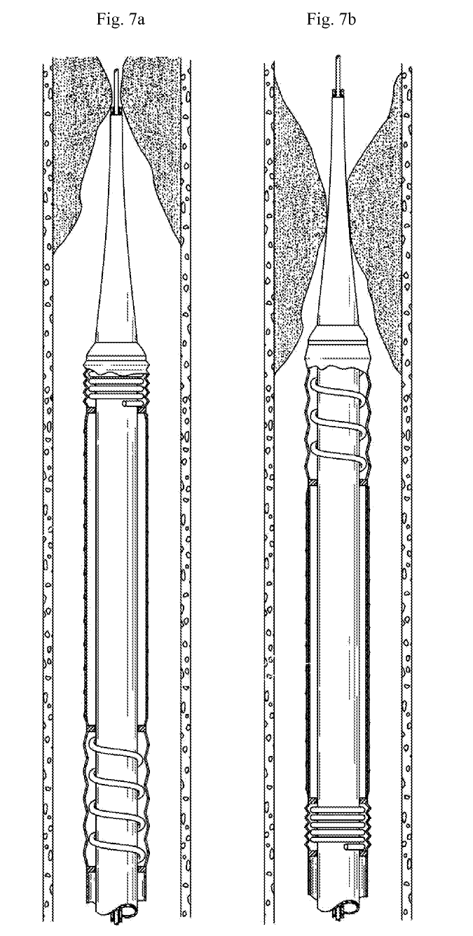

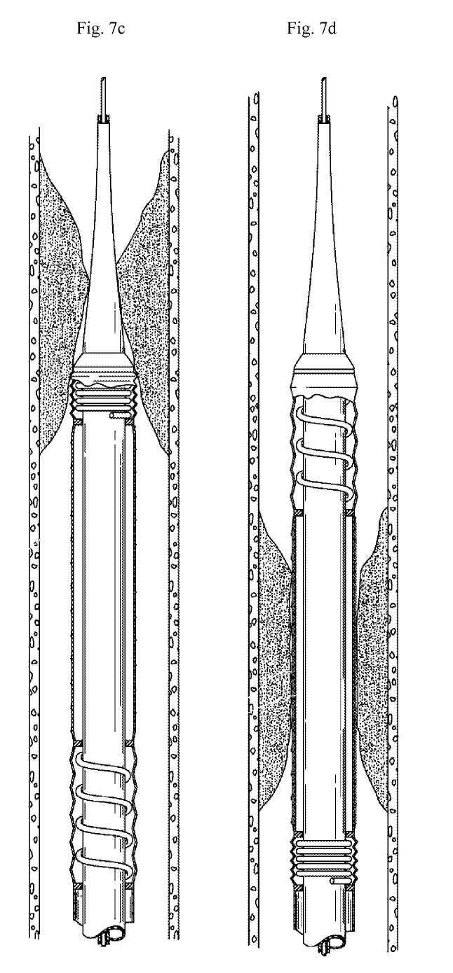

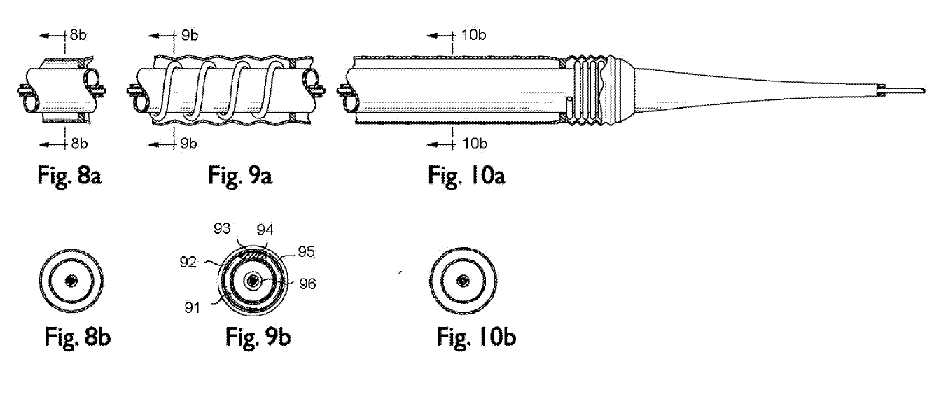

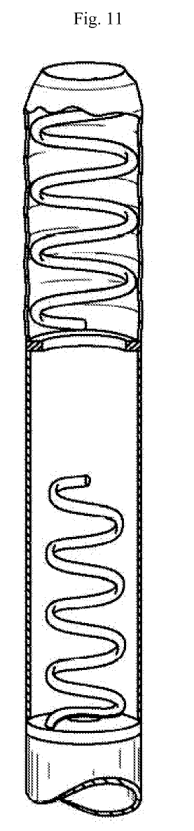

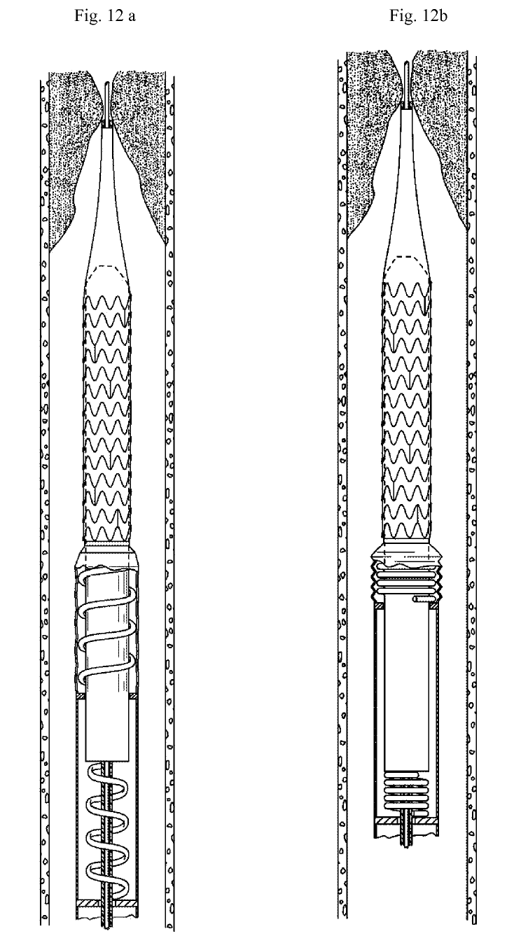

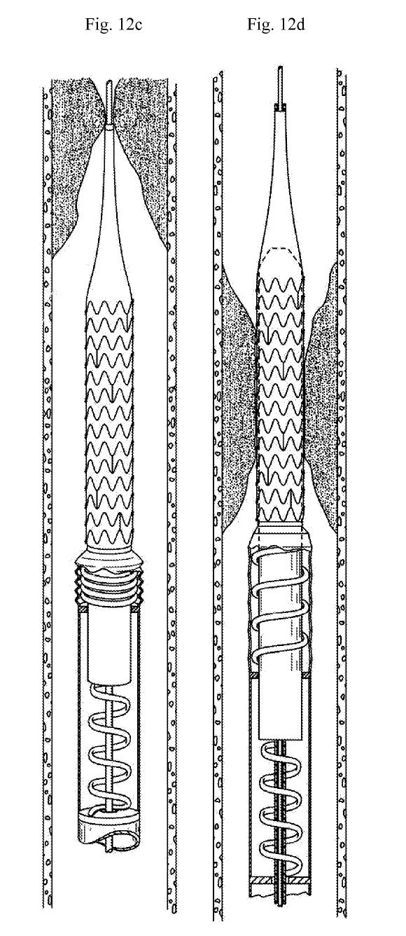

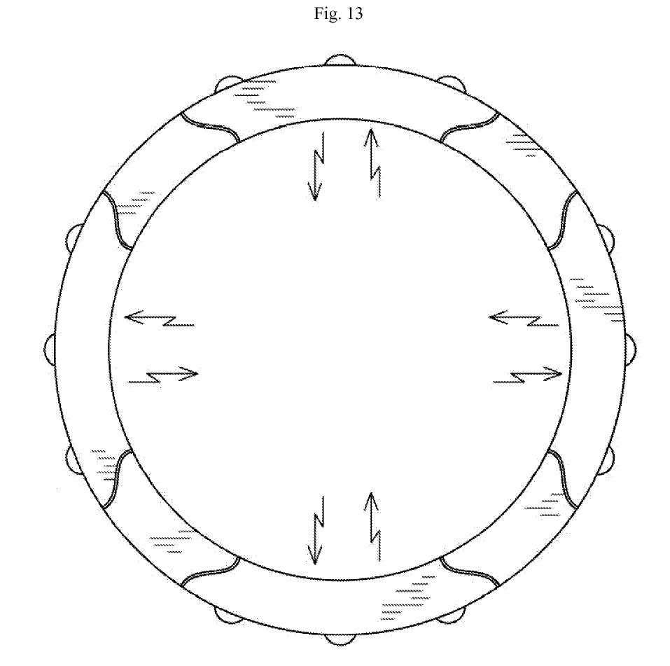

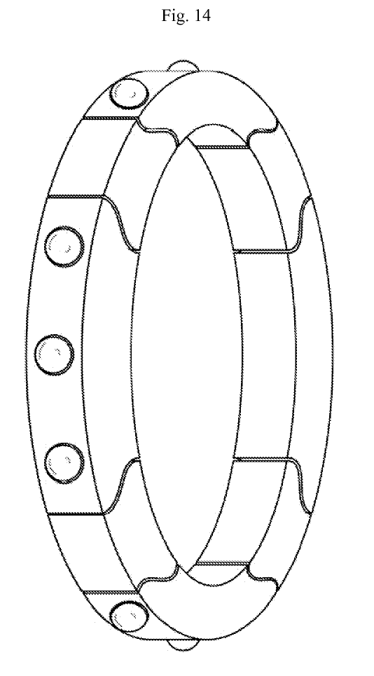

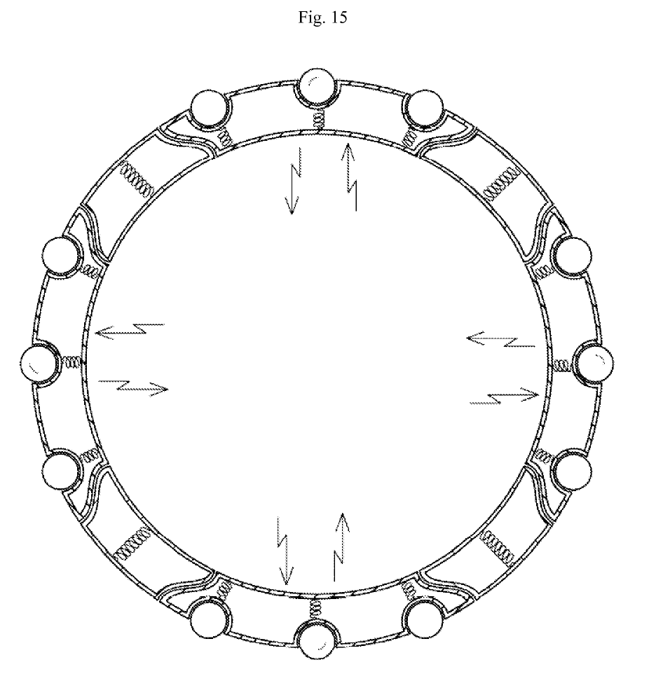

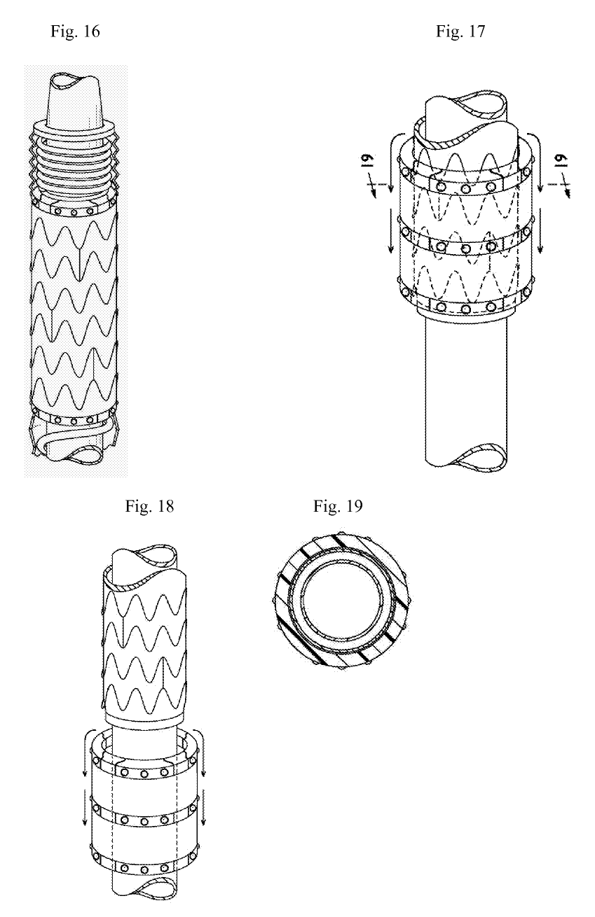

In other preferred embodiments, the system comprises a single, proximal accordion spring segment. In one preferred embodiment, the single proximal accordion spring segment is as depicted in FIGS. 1 (a-e) through 4 (a-b). In some preferred embodiments, the system comprises a primary “pushing” spring system comprising a primary elongate tubular catheter system with a pushing spring component disposed just proximal to the balloon-stent assembly. An object of the design of this system is to provide a proximal pushing element that is continuous with “accordions” against the balloon-stent distal assembly. In preferred embodiments, the spring component is comprised of a helical wound spring in an expanded “relaxed/unloaded” state. The spring segment is in continuity with the entire catheter shaft but the spring section is interposed between the more proximal aspect of the catheter and the distal balloon.

In one preferred embodiment the spring segment has a primary pushing plate spring attachment which is therein connected to an “accordion-concertina” spring which then attaches to the proximal edge of the balloon-stent assembly at a stent abutment plate. The spring may be comprised of stainless steel or may be comprised of a polymeric material such as polyethylene (PE), polypropylene (PP), polyvinyl chloride (PVC) or polytretrofloroethylene (PTFE). The spring may be formed of any suitable material that would provide appropriate structure reinforcements such as stainless steel flat wire or biologically compatible metals, polymer, plastics, alloys or composite materials. The spring is formed by a portion of a wire that has a proximal end and a distal end.

The helical wound spring itself dwells within the central catheter balloon inflation lumen which serves to facilitate inflation and deflation of the balloon for dilatation of vascular lesions, as well as for balloon mediated deployment of stents. The balloon lumen may be in communication with the catheter shaft as in traditional catheters via a separate balloon inflation lumen that accommodates the movement of inflation fluid under pressure from an inflation-deflation device operated by and connected to from outside the body at the most proximal end of the catheter, to distal at the balloon end. The coil is secured in the flexible elongate tubular member just proximal to the proximal extremity of the balloon and permits passage of the inflation medium for the balloon from the lumen into the anterior of the balloon. The proximal end of the wire is attached to a circular abutment pushing plate attached to the catheter shaft within the balloon inflation lumen and encircling the guide wire shaft lumen. The wire is helically disposed about the inner shaft from a point proximal to the distal end. At its distal end, the spring wire connects to a distal push plate that is positioned just proximal to the balloon segment. Although the particular embodiments depicted in the figures utilize a helically disposed spring coil to bias the balloon-stent component of the catheter distally through a stenosis, other configurations are possible without deviating from the scope of the invention.

The pushing spring component is covered by an outer sheath accordion envelope that acts as the outer barrier of the catheter but through its accordion motion allows the pushing spring/outer sheath to accordion/contract/load against the balloon-stent component. The pushing spring component is characterized internally by an inner sheath accordion segment that forms the guide wire shaft, that acts as the inner accordion spring component through its accordion/contracting motion that allows the pushing spring/outer sheath to accordion/contract/load over the innermost guidewire and ultimately against the balloon-stent component. The catheter assembly has an inner lumen/channel inserted over a guide wire (embodiments are designed to accommodate both monorail rapid exchange configuration and/or “OTW” configurations).

Operationally, the entire catheter assembly is pushed from the proximal portion of the catheter outside the patient and advanced over the guide wire. If no significant resistance is encountered, the entire shaft moves as a single piece without any contraction of the accordion spring segment and the balloon-stent assembly delivered to the desired lesion location within the arterial system. If, however, any resistance is encountered throughout its course through the vasculature, the resistance most likely will be encountered at the balloon-stent assembly, the stiffest, most inflexible portion of the distal catheter. By virtue of the design, when resistance is encountered at the balloon-stent assembly, this component of the system will slow down or stop within the resistance point and, as the proximal catheter is pushed against this resistance, traction will be gained which will allow the proximal catheter to push against the primary spring segment which “accordions”. As the accordion segments compress, including both accordion shortening of the inner guide wire lumen shaft accordion component as well as the outer accordion shaft component, this contraction of these two accordion shafts facilitates compression from the push plate to “load” the spring. The simultaneous contraction or shortening of the inner guide wire accordion shaft element and outer sheath accordion component together with the spring, constitute a plunger spring motion which builds up energy within that accordion spring segment which is then transmitted to the more distal abutment plate interfacing with the balloon-stent assembly, the force thereby pushing the balloon-stent forward over the inner most guide wire through the resistance point within the blood vessel. As the spring releases sufficient force to propel the balloon-stent section forward over the guide wire, the accordion envelope can then expand to its “resting” state. This motion can be repeated over and over to advance the catheter.

In other preferred embodiments of this accordion spring single shaft system, the inner core accordion component is attached to telescoping tubes, rods or struts longitudinally disposed from this proximal pushing segment through the balloon-stent segment to attach most distally to a second accordion spring segment that is positioned distal to the balloon stent assembly and connecting to the distal end of the catheter, which has an accordion spring itself. This configuration allows for a dual spring system: proximal pushing spring and distal pulling spring. In some embodiments, the distal accordion segment is structured similarly the proximal segment previously described, including an inner core accordion guide wire shaft component and an outer core primary catheter shaft accordion component. In some embodiments, the distal spring is designed similarly to the proximal spring with inner and outer accordion elements and a wound spring in between the 2 accordions sheaths; the spring in its resting state is contracted and connects to the distal end of the balloon with an abutment plate. The telescoping rods dwell within the balloon lumen disposed circumferentially exterior to and around the guide wire shaft; the tubes extend from the proximal spring abutment plate through the balloon segment to the distal spring.

Catheters of these dual spring embodiments operate similar to the previously described proximal spring single shaft design. In some embodiments, the distal segment comprises a relaxed, contracted spring at rest. When resistance is encountered as the proximal accordion spring is advanced and contracted, as described above, the shortening of the proximal spring not only exerts pressure forward on the balloon-stent assembly, such spring shortening also concurrently advances the longitudinally disposed telescoping rods from the proximal accordion spring, through the balloon stent assembly, to the distal accordion spring. These hydraulic or telescoping tubes advance as the proximal spring is contracted. As the longitudinal rods advance distally, they expand the distal spring which creates a pulling force to facilitate advancement of the balloon-stent assembly through the vascular resistance. The combination of the proximal and distal springs creates a “push-pull” proximal-distal springs system, whereby the initial proximal pushing of the contracting spring exerts primary direct push force to advance the balloon-stent assembly and simultaneously through advancement of the longitudinal rods expands the distal spring which provides a distal pulling force. The two springs operate in concert providing a dual push-pull mechanical system to facilitate movement of the balloon-stent assembly through the vascular resistance.

In some embodiments, the distal accordion pull segment comprises, as its pulling multiplier force, elastic bands (or similar elastic materials) disposed from the very distal pulling end of the distal accordion segment and attached to the very distal end of the balloon-stent assembly. The motion of the distal spring over the wire exerts a pulling force on the elastic elements which facilitates tugging or pulling of the balloon stent assembly forward through the vascular pathway.

Some embodiments comprise a primary “pushing” spring catheter system comprising a primary elongate tubular catheter system which functions as the primary driving catheter shaft that telescopes within an outer slidable balloon-stent delivery system disposed external to the primary catheter shaft. The primary goal of the design of this system is to provide a proximal inner pushing element within a slidable outer core stent balloon component, whereby between the two systems are interconnected springs interposed either singly or in series.

Some embodiments comprise a primary catheter shaft serving as the inner driving rod or ram. Circumferentially disposed external to the catheter shaft ram is the balloon-stent assembly. An accordion spring component connects between the primary catheter shaft and the balloon-stent assembly. The coil spring is interposed between a push plate attachment proximally and a stent abutment plate distally and this spring section of the catheter is covered by an accordion telescoping segment.

The primary catheter shaft is disposed as an inner component pushing spring that acts as the generating force pushing the outer over-riding balloon-stent system. In this embodiment, the catheter shaft acts as an inner compression member comprising an elongated pusher bar that helps to push the spring forward to facilitate forward motion of the balloon-stent. The primary catheter shaft inner driving rod/ram is an extension of the primary catheter shaft and circumferentially disposed external to this ram is the balloon-stent assembly. In this configuration, the stent balloon assembly slides back and forth along the outer circumference of the primary catheter shaft with an accordion like spring connection between the distal extending inner rim of the primary catheter shaft at which proximal end is a stent abutment plate connected more proximally to a push spring thereby attached to a more proximal pushing plate spring attachment which is all in assembly contained circumferential to the inner driving ram. The coil spring is interposed between a push plate attachment proximally and a stent abutment plate distally and this section of the catheter is covered by an accordion telescoping segment. The inner catheter shaft-pushing spring component is covered by an accordion envelope that acts as the outer barrier of the catheter but through its accordion motion allows the catheter to ride inside of (contract against) the outer balloon-stent assembly.

Other embodiments of this configuration relate to the system and method by which the primary catheter shaft balloon inflation lumen is attached to and inflates the over-riding balloon. To accommodate accordion motion of the spring segment to push the balloon, yet to achieve continuity with the proximal catheter shaft to inflate the balloon, two systems may optionally be used. In one preferred embodiment, the primary catheter shaft balloon lumen terminates distally in a telescoping tube design in which the downstream tube component is integrated within the balloon, thereby allowing sliding or telescoping motion to push the balloon but maintaining fluid continuity with the balloon to inflate it. In another embodiment, the balloon may be attached by a hose tether that passes through the inner core of the spring system and has sufficient length to accommodate the longitudinal movement of the inner driving balloon-stent rod system. In another embodiment, the spring itself is constructed of an inner helically shaped tube that allows a hydraulic connection between the catheter balloon lumen and the balloon itself. In still another embodiment, the stent segment is covered by and constructed with a self-expanding sheath system that does not require a balloon.

The catheter shaft is pushed from outside of the patient through a guiding catheter into the desired circulation. As it is advanced in the vessel, when resistance is encountered at the balloon-stent assembly level, traction at the point of resistance builds tension as the inner driving ram pushes through the inner lumen of the balloon-stent assembly, which then exerts force against the push plate spring abutment thereby upon the pushing spring which then accordions loading the spring and, once the inner ram is driven further forward, allows the tension within the spring to push the balloon-stent assembly forward sliding coaxially over the inner catheter shaft ram.

In some embodiments, the accordion telescoping spring segment is disposed distal to the balloon-stent assembly. As the balloon-stent encounters the resistance/friction of a stenotic lesion, further advancement of the catheter shaft-inner ram extends the distal spring attached to the balloon-stent by an abutment plate; this inner core ram loads the distal extending spring which then pulls the balloon-stent assembly forward coaxially over this inner telescoping ram. As the primary catheter drives the inner ram coaxially through the outer core balloon-stent assembly, the balloon-stent which is caught within the friction of the lesion allows the inner ram to extend this distal spring attached on either side abutment plates. The inner core spring ram loads the distal extending spring which then pulls the balloon-stent assembly forward coaxially over the inner telescoping ram. In a related additional embodiment, the catheter has both proximal and distal spring mechanisms disposed both proximal to the balloon-stent assembly and distal to the balloon-stent assembly with the inner telescoping ram as it is extended by contracting the proximal spring loading tension to push the stent balloon system forward, as well as the inner core advancement extending the distal spring which pulls the balloon-stent assembly forward. In aggregate, this creates a push pull system of dual spring mechanisms.

The spring systems, whether proximal or distal and whether an inner core shaft or primary shaft delivery systems, may be actuated by non-manual systems including mechanical systems that are hydraulic, magnetic, pneumatic, or of other mechanical power systems.

In some embodiments, the system comprises a primary “pushing” spring system which comprises a primary elongate tubular catheter system with a distal inner core slidable balloon-stent rod delivery system. An object of the system is to provide a proximal pushing element and a slidable inner core stent balloon rod delivery system, whereby between the two systems are interconnected springs interposed either singly or in series. In one preferred embodiment, the proximal outer primary catheter shaft has at its distal end a backstop plate connected to a backstop spring that interconnects with the proximal end of the inner pushing balloon-stent rod. The stent rod/stylet is housed within the lumen of the primary proximal catheter and the proximal catheter adjoins to the stent balloon system through a primary pushing plate spring attachment which is therein connected to an “accordion-concertina” spring which then attaches to the proximal edge of the balloon-stent assembly at the stent abutment plate. In its operational form, the most distal end of the entire catheter assembly has an inner lumen/channel inserted over a guide wire (preferably a monorail rapid exchange configuration, or in an “OTW” configuration). The outer sheath pushing spring component is covered by an accordion envelope that acts as the outer barrier of the catheter but through its accordion motion allows the outer sheath to ride over (contract against) the inner balloon-stent rod component. To facilitate inflation and deflation of the balloon for dilatation of vascular lesions and when used for balloon mediated deployment of stents, the balloon lumen may be in communication with the catheter shaft as in traditional catheters via a separate balloon inflation lumen that accommodates the movement of inflation fluid under pressure from an inflation-deflation device operated by and connected to from outside the body at the most proximal end of the catheter, to distal at the balloon end. Alternatively, to accommodate accordion motion of the spring segment the balloon may be attached by a hose tether that passes through the inner core of the spring system and has sufficient length to accommodate the longitudinal movement of the inner driving balloon-stent rod system.

The catheter is advanced over the guide wire and if no resistance is encountered, the entire assembly is pushed from the proximal portion of the catheter outside the patient with the delivery of the balloon-stent assembly system to the desired lesion location within the arterial system. If, however, any resistance is encountered throughout its course through the vasculature, the resistance most likely will be encountered at the stiffest, most inflexible portion of the distal catheter which typically will be the balloon-stent assembly. By virtue of the design, when resistance is encountered at the balloon-stent assembly, this component of the system will slow down or stop within the resistance point and, as the proximal catheter is pushed against this resistance, traction will be gained which will allow the proximal catheter to push against the primary outer shaft spring-balloon stent interface thereby “loading” the primary spring, which action by itself will generate spring force to facilitate “pushing” the stent balloon assembly forward. By virtue of the inner core stent rod design, as the balloon-stent assembly slows down or gains traction within the stenotic resistance section, the inner core including the balloon-stent assembly will be pushed back into the primary catheter lumen recess, thus resulting in an action which will load the backstop spring connecting the proximal end of the inner core stent rod to the backstop plate within the primary catheter; this action will thereby providing loading of both the backstop spring as well as the primary push spring. As these springs both singly and in aggregate are “loaded,” which occurs with the inner rod being pushed to retract within the outer core shaft lumen, once sufficient tension is developed within the springs the inner core rod will be pushed forward through the resistance frictional segment.

The function and operation of the accordion envelope covering the outer sheath spring is as follows: As the outer sheath spring pushes against resistance built up by the balloon-stent rod assembly until the spring releases sufficient force to propel the inner balloon-stent rod forward and the encircling/covering accordion envelope can then expand to its “resting” state.

A preferred embodiment of this system also includes a catheter with a plurality of springs placed in series along the proximal and/or distal component of the catheter. Such serial springs may be engaged, loaded and released simultaneously as with the single spring mechanism previously described. Alternatively, this multiple “springs in series” design also has a unique design to sequentially engage, load and release from most distal to most proximal loading and most proximal to distal release to apply forward propulsive force in a peristaltic fashion that would facilitate progressive advancement of the distal component of the catheter through a resistant circulation by this series of springs engaged sequentially and released sequentially. This sequentially released series of springs includes a novel and unique control mechanism similarly controlled by the operator outside of the body at the most proximal end of the catheter.

In some embodiments, the method and system relates to a movable core coronary guide wire system, and more particularly relates to a guide wire having an inner core and an outer core that move axially within each other. The movable core is constructed whereby the very distal inner core of the wire is approximately 0.010-0.014 inches in diameter and has a very steerable floppy atraumatic tip for traversing the coronary anatomy. The outer core consists of a thicker, approximately 0.014-0.018 inches, more rigid construction that telescopes over the extended inner core. The outer core has the ability to connect to and pull the distal end of a balloon-stent catheter, such as the spring balloon-stent systems. In this design, the connection between the movable core that attaches to and “tugs” the distal spring/balloon-stent assembly may be various methods and designs including magnets, automatic latches or other types of connections whereby the catheter is moved over the movable core wire component and automatically engages the movable connecting site with sufficient strength of connection to allow the forward motion of the movable core to load the distal spring and pull the catheter through resistance points.

The entire inner core/outer core system may be assembled together outside the patient. Then the outer core is inserted into a Y arm attachment proximal to the guiding catheter through an 0-ring with the outer core advanced with the inner core retracted and the outer core advanced to the end of the guiding catheter but remaining within it. The 0-ring can then be tightened down around the outer core which serves as the seal with the 0-ring. The inner core point 0.014 inch wire can then be maneuvered down the coronary artery past and distal to the culprit lesion. The outer core may then be advanced over the inner core so that the portion of the outer core wire is distal to the stenosis, but the component of the outer core wire with the magnetic flange interlock for the balloon stent catheter is maintained proximal to the lesion. This wire position can provide a maximum stiffness prior to inserting the catheter into the patient. The wire can also then serve for the “tug” pulling system to facilitate advancement of the balloon stent assembly through a tight stenosis. Once the wire is in place, the catheter can then be advanced through the blood vessel. The core wire may lie within the distal portion of the catheter, initially in part to support the catheter distal region against buckling. Once the balloon catheter is advanced over the wire and distal to the guide catheter, if the balloon catheter encounters resistance to further passage, the core wire is retracted slightly relative to the balloon catheter and when the component of the outer core wire is positioned adjacent to the balloon catheter magnetic interlock zone, the flange of the outer core is activated and interlocks with the magnetic connection of the balloon catheter. The outer magnetic core can then be advanced over the inner core in order to facilitate downstream pulling or “tugging” to pull the balloon-stent system through the stenosis. As the outer core is advanced and provides a tugging force on the balloon, the balloon can itself be advanced together with this outer core giving it maximal pushability and pullability through the stenosis. In one method, the outer core wire is advanced over the inner core wire distally and this tugs the balloon-stent assembly through the stenosis.

In one embodiment, this connection may involve a slidable distal component within the balloon catheter that is attached to mini-pulleys that can be pulled by the outer core producing a pulley-tug system with elastic connections that then pulls the distal end of the balloon through the stenosis. In another embodiment, the inner core wire is inserted through the guide catheter and positioned distal to the target lesion. Then the balloon catheter is loaded on the indwelling inner wire, but prior to insertion into the guide, the outer wire is inserted from the back end of the inner wire to connect together with the distal end of the balloon catheter. Then the entire balloon catheter and outer wire assembly are advanced into the guide catheter and down the target vessel. If resistance is encountered, the outer guide wire can be advanced over the inner guide wire to “tug” or pull the distal end of the catheter forward over the wire.

This movable core wire support and pulling system can be incorporated with all of the previously described catheter systems.

Another preferred embodiment of the system for propulsion based vascular device delivery is a wheel enhanced catheter cylinder or sleeve device designed to enhance maneuverability through the vascular terrain. In this embodiment, portions of the catheter delivery system in general, and the balloon-stent assembly in particular, are equipped with wheel assisted catheter tracking cylinder bearings or sleeves.

In one preferred embodiment, the catheter is fitted with special compressible soft wheel cylinders with rolling, floating, soft deflecting cylinder bearings. These cylindrical bearings are designed to fit on the proximal and distal margins of the balloon-stent delivery system. These cylinders are designed to be low profile with only the minimal aspect of the bearing wheels extending beyond the maximal catheter circumference. The catheter bearings-wheels are disposed circumferentially around these soft compressible rings in a symmetrical fashion. These bearings/wheels are made of soft compressible rubber or similar silastic atraumatic and lubricous material. These bearings/wheels spin with minimal friction within microaxials or other similar structures that allow them to freely rotate and thereby help propel the cylinder portion and therefore the entire balloon-stent assembly through high-friction zones. These bearings/wheels may be attached singly or in subcomponents of three or less or more onto compressible springs that act as “shock” absorbers. The embedded “shock” absorbers, ball bearing trackers, allow the segment that encounters resistance to spin and float in and out to accommodate the movement of the segment through friction zones. Optionally, these cylinder components may also be articulated to allow the entire system to “flex” its way through such friction zones.

In another preferred embodiment, the cylinder bearing/wheels are disposed around a low profile sheath that covers the balloon-stent assembly and allows the balloon-stent assembly to be advanced through high-friction zones, the structure of some components of the cylinders are similar but are attached in one or a plurality of cylinders within a low profile sheath that, once its positioned with the target zone, can be retracted to allow the balloon-stent assembly to expand and deploy.

These spring systems, whether proximal or distal and whether an inner core shaft or primary shaft delivery systems, may be manually actuated by simple advancement of the catheter shaft. In some embodiments, the spring systems may be connected to and actuated by non-manual mechanisms including mechanical systems that are hydraulic, magnetic, pneumatic, or other mechanical or remote power systems. In such designs, the unique novel control actuation mechanisms serve to engage and disengage the springs with a control mechanism placed at the proximal end of the catheter outside the body to be controlled by the operator. The mechanical operating handle may comprise any mechanical, electromechanical, pneumatic or hydraulic handle configurement in communication with, directly or indirectly through intervening ports, the distal portions outer guide channel member. Communication would include, by way of illustration and not by way of limitation, a handle that uses, or is otherwise associated with, directly or indirectly, an elongated mechanical wire, rod, shaft, cable, sheath, pneumatic tube or hydraulic pistons, cylinders and/or flow pass configured removing the outer guide channel member proximally in order to activate the spring or other force generating system.

One embodiment of a mechanically assisted propulsion system includes a hydraulically driven spring system in which the spring(s) are loaded/unloaded through the action of hydraulically activated-driven push plates/pistons or other devices that couple to the springs and compress-load or extend load them, thus providing the force for forward catheter movement. These hydraulic systems may be extensions of those already designed into the catheter for fluid driven expansion of the dilatation balloon, or comprise a dedicated additional lumen/microtubes designed for spring activation. These hydraulic systems can be driven on the proximal end outside the body by pressure driven fluid. Alternatively, the hydraulic systems may be pneumatically driven by compressed gases such as carbon dioxide, delivered through an analogous system of lumens or microtubes.

Alternatively, these systems can be driven on the proximal end outside the body by pneumatically driven compressed gases such as carbon dioxide, delivered through an analogous system of lumens or microtubes.

In another embodiment, the spring systems may be activated/driven by rotational cable attached at the distal end to a rotational transducer plate at the interface between the cable and the spring, this transducer interface transmitting the torque power/force of the rotating cable to a rotating spring (like in a watch) which is attached to and transduces the force to the push (or pull) spring. The rotating cable may be attached at its proximal end to a catheter based rotation device, such as is known in the field of interventional Cardiology for use in catheters such as those employed for rotational atherectomy or for Infrared catheters

Another preferred embodiment of the system for propulsion based vascular device delivery is a magnetic based wire-catheter-device designed. A preferred embodiment of this design is a variable magnetized wire which serves as the rail as well as the propulsive power force of this device system. The wire may be conventional (eg 0.014 to 0.018 inches in diameter) or miniaturized and has a flexible atraumatic distal component. The wire is constructed of a series of linearly placed magnetic sections, which can be sequentially and linearly activated to provide a linearly directed magnetic pulling force designed to act upon a magnetically sensitive over-the-wire delivery device. The sequential magnetic component control device is located at the most proximal end of the catheter outside the body and can be connected to the wire component that is most proximal and outside of the body and through this magnetic controller the linearly arranged sequential components of the magnetic wire can be activated. In one preferred embodiment of the invention, the wire is designed as a flexible steerable wire that can be primarily advanced through a target circulation as with any other vascular guide wire.

Alternatively, the magnetic wire can be placed through a previously positioned guide wire system with the primary guide wire constituted by a traditional vascular guide wire that is steered through the target in circulation with a exchange or delivery catheter advanced over the primary wire, the primary wire removed from the delivery-exchange catheter lumen, and the magnetic wire then passed through the exchange catheter lumen and positioned in the distal circulation without the necessity of steering this magnetic wire through the entire circulation primarily.

One preferred embodiment of this magnetic vascular device system is a balloon dilatation catheter (suitable for both primary dilatation of obstructed vascular segments as well as delivery of balloon expandable devices such as stents) in which the distal end comprises a series of magnetic sensitive metallic components that are designed to associate with and interact with the uniquely designed multi-component magnetic wire. These magnetic components are located on the inner surface (luminal surface) of the balloon catheter as its most distal ends. The catheter would be advanced over the magnetic wire with initial hand advancement of the catheter over the wire in a traditional over-the-wire fashion. At the desired point in the circulation, particularly those at which there is resistance to further mechanical advancement, the sequential magnetized multi-component system would be activated by the external magnetic controller which would then sequentially activate the magnetic components of the wire in a fashion that would sequentially advance the catheter distal component through the resistance portion of the circulation; this magnetized pulling force would be aided by this simultaneous operator hand advancement of the catheter to produce a facilitated mechanical and electromagnetic catheter advancement. This magnetic system could be well suited for the movable core wire designs discussed previously.

An object of the invention is to achieve designs that would be suitable for both traditional disposable catheter systems, but also reusable systems in which the catheters once used could be re-sterilized and re-used, as well as those with intent for stent delivery systems to be resterilized and have new stents mounted upon them for re-use.

It is the intent of the design to facilitate all of these novel systems to be miniaturized (ultimately into micro-machines and finally into nano-machines). This refers to all of the previously described spring systems, both manual and mechanically powered. The miniaturized approach is also especially applicable to the magnetic system, which is suitable for an ultra-miniaturized barrier and/or catheter system, whereby the magnetic pulling propulsive force combined with hand advancement would allow for greater tractability, pushability and therefore deliverability of the catheter system, thus allowing downsizing of the catheter size as the body of the catheter would not need to be so large and provide the previously necessary external pushing forces.

An additional preferred embodiment of this magnetic vascular device delivery system is ultra-ultra(nano) miniaturization with miniaturized balloons on filamentous tethers (with or without mounted downsized miniaturized stents). In this ultra-ultra miniaturized system, the catheter itself would be eliminated and dilatation balloon-stent deployment systems would be designed on filamentous tethers (with or without mounted downsized miniaturized stents). Such systems may be driven by non-manual mechanical methods including intrinsic nano-miniaturized microfluidics, pneumatics and magnetic components integrated into the catheters and/or filament delivery tubes. In one preferred embodiment, the miniaturized inflatable balloon would be attached by a filamentous hollow tube wire or miniaturized tube which is controlled outside of the body. The free balloon component with intraluminal surface magnetic device would be advanced over the external portion of the magnetic wire and then pulled by the sequential magnetic forces along the entire course of the wire into the distal target circulation and positioned at the desired target location. The balloon could then be inflated through the filamentous balloon dilatation tube (wire) connected to the balloon lumen and controlled through an external inflation device that is uniquely designed for this filamentous tube connected free balloon catheter. Such free balloon systems can also be utilized with unique stent deployment designs in which a metallic stent would be mounted on this free delivery balloon and similarly advanced by magnetic controls through the circulation with balloon dilatation resulting in stent ultimate delivery as well as deployment of the stent. This unique design facilitates miniaturization of the balloon and stent with substantial downsizing of its caliber and improved flexibility which would thereby translate into improved tractability and deliverability of the devices to the target circulations, resulting in further miniaturization of the devices and downsizing of various catheters into the circulations. It can also be used to access very difficult circulation such as the very distal cerebral circulation.

Although the foregoing detailed description of the present invention has been described by reference to an exemplary embodiment, and the best mode contemplated for carrying out the present invention has been shown and described, it will be understood that certain changes, modification or variations may be made in embodying the above invention, and in the construction thereof, other than those specifically set forth herein, may be achieved by those skilled in the art without departing from the spirit and scope of the invention, and that such changes, modification or variations are to be considered as being within the overall scope of the present invention. Therefore, it is contemplated to cover the present invention and any and all changes, modifications, variations, or equivalents that fall with in the true spirit and scope of the underlying principles disclosed and claimed herein.

What is claimed is:

1. A catheter system for delivery of a balloon or balloon stent assembly comprising: a mechanical means for advancing the assembly through an area of vascular resistance.

2. The system of claim 1, wherein the mechanical means for advancing the assembly through an area of vascular resistance comprises at least one coil spring disposed proximally of the balloon or balloon stent assembly.

3. The system of claim 1, wherein the mechanical means for advancing the assembly through an area of vascular resistance comprises at least one coil spring disposed distally of the balloon or balloon stent assembly.

4. The system of claim 1, wherein the mechanical means for advancing the assembly through an area of vascular resistance comprises at least one coil spring disposed both proximally and distally of the balloon or balloon stent assembly.

5. The system of claim 1, further comprising a telescoping shaft having an inner tube and an outer tube.

6. The system of claim 5, wherein the mechanical means for advancing the assembly through an area of vascular resistance is disposed along one of said inner tube or said outer tube.

7. The system of claims 1, wherein the system further comprises a moveable core guide wire.

8. The system of claim 1, wherein the system further comprises a series of wheels or bearings.

9. The system of claim 1, wherein the system further comprises a pulley system.

10. The system of claim 1, wherein the system is driven mechanically by hydraulic, pneumatic, magnetic, or cable rotation means, or any combination thereof.

11. The system of claim 1, wherein the system is sterilized before reuse.

12. The system of claim 1, wherein the crossing profile of the system is in the range of 1 to 1000 micrometers.

13. The system of claim 1, wherein the crossing profile of the system is in the range of 1 to 1000 nanometers.
